(19) United States
(12) Patent Application Publication
(10) Pub. No.: US 2003/0012824 A1
(43) Pub. Date: Jan. 16, 2003

(54) Orally administered anti-stress composition

(76) Inventors: Dana Beth Ott, Comstock Park, MI (US); Robert Henry Dobberstein, Lincoln, NE (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/898,140

(22) Filed: Jul. 3, 2001

(51) Int. Cl. A61K 33/42; A61K 33/08; A61K 35/78

(52) U.S. Cl. 424/602; 424/725; 424/690; 424/750; 424/756; 424/745; 424/764; 424/737

(57) ABSTRACT

The present invention relates to an orally administered composition to relieve stress comprising from about 100 mg to about 2000 mg of at least one antacid, about 50 mg to about 500 mg of at least one anxiety reducing compound wherein the amount of anxiety reducing compound is based on a concentrated extract containing not less than 0.5% of the essential oil of the respective anxiety reducing compound, and about 20 mg to about 600 mg of at least one mental alertness inducing compound wherein the amount of the mental alertness inducing compound is based on a concentrated extract of the respective mental alertness inducing compound. The anti-stress compositions simultaneously provide the user with a considerable sense of well-being by providing relief from excess gastrointestinal acidity, reducing anxiety, and increasing mental alertness or thinking ability. The overall sense in the user is that stress is reduced emotionally as well as physically as a result of the effects of the present invention.

### FIELD OF THE INVENTION

The present invention relates to an orally administered anti-stress composition comprising a critical amount of an antacid, anxiety reducing compound, and mental alertness inducing compound. The anti-stress compositions simultaneously provide the user with a considerable sense of well-being by providing relief from excess gastrointestinal acidity, reducing anxiety, and increasing mental alertness or thinking ability.

### BACKGROUND OF THE INVENTION

Stress is a condition that adversely affects humans and animals. The effect of stress can be exhibited in a variety of ways. For example, often there is a general decrease in overall health and well being. This may manifest itself in weight loss, weight gain, deteriorating overall health, and/or an increased susceptibility to viral and bacterial infections.

There are numerous predictable times during life where stress is likely to increase. The most common occurrences are stress prior to a test, business meeting, court appearance, or public speaking engagement. Consequently, reducing stress at such times should result in improved behavior and performance.

Stress does not only affect adults. Stress related behavioral problems also manifest themselves in children, including preschool children. For example, a study recently published in the Journal of the American Medical Association examined the medical records of more than 200,000 preschoolers, ages 2 through 4, from 1991 to 1995. The study found that the use of RITALIN® to treat attention deficit hyperactivity disorder doubled over that period, as did the number of preschoolers on anti-depressants such as PROSAC®. The increase in the number of preschool children being prescribed psychiatric drugs to treat behavioral problems is especially troubling because it may suggest an overuse of harmful drugs to solve behavioral problems that do not require such strong medication. Of course, the diagnoses of mental disturbances that might require drug therapy are difficult to make with any certainty in very young children, however, there is a need for safer medications to treat humans and animals suffering from stress.

### SUMMARY OF THE INVENTION

The present invention provides an orally administered composition to relieve stress comprising from about 100 mg to about 2000 mg of at least one antacid, about 50 mg to about 500 mg of at least one anxiety reducing compound wherein the amount of anxiety reducing compound is based on a concentrated extract containing not less than 0.5% of the essential oil of the respective anxiety reducing compound, and about 20 mg to about 600 mg of at least one mental alertness inducing compound wherein the amount of the mental alertness inducing compound is based on a concentrated extract of the respective mental alertness inducing compound.

The principal advantage of the anti-stress compositions are that the user can obtain relief from excess gastrointestinal acidity, reduction in anxiety, and increased mental alertness simultaneously from the same composition. These three actions combined produce in the user a considerable sense of well-being for three reasons. First, the user's abdominal discomfort is diminished. Second, the effects of the anxiety reducing compound works to provide additional calm. Third, the increased blood circulation improves the general health of the affected individual and mental alertness or thinking ability. The overall sense in the user is that stress is reduced emotionally as well as physically as a result of the effects of the present invention.

### DESCRIPTION OF THE INVENTION

The anti-stress compositions of the invention are orally administered to humans or animals in solid or liquid form. The anti-stress compositions are not harmful and generally recognized as safe (GRAS) and may be administered to children including babies. Suitable liquid forms include, but are not limited to, solutions, emulsions, and suspensions. Suitable solid and semi-solid forms include, but are not limited to, capsules, caplets, powders, and tablets, and include soft and hard, chewable and non-chewable forms. Specific examples of solid or semi-solid forms include gels and food-like systems such as bars and candies.

The anti-stress compositions contain an antacid, an anxiety reducing compound, and a mental alertness inducing compound. More than one antacid, anxiety reducing compound, and/or mental alertness inducing compound may be used in the anti-stress compositions. Examples of antacids suitable for use herein are any antacids acceptable to the Food and Drug Administration, such as aluminum carbonate, aluminum hydroxide (or as aluminum hydroxide-hexitol stabilized polymer, aluminum hydroxide-magnesium hydroxide codried gel, aluminum hydroxide-magnesium trisilicate codried gel, aluminum hydroxide-sucrose powder hydrated), aluminum phosphate, aluminum hydroxy carbonate, dihydroxy aluminum sodium carbonate, aluminum magnesium glycinate, dihydroxy aluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, magnesium trisilicate, sodium bicarbonate, potassium bicarbonate, glycine, dried milk solids, sodium carbonate, potassium carbonate, and sodium potassium tartrate. A combination of antacids may also be used. Preferably, the antacid is selected from aluminum hydroxide, calcium carbonate, magnesium carbonate, and magnesium hydroxide.

The antacid is present in the anti-stress composition in an amount to neutralize at least 5 milliequivalents of gastrointestinal acid. Preferably, the antacid is present in an amount of from about 100 mg to about 2000 mg, more preferably from about 200 mg to about 1200 mg. Most preferably, the antacid is present in an amount of from about 400 to about 800 mg.

The anxiety reducing compound is selected from herbal compounds and nonherbal compounds which exhibit anxiety reducing activity. The herbal compound may be in the form of ground plant or parts of plant, liquid extract of plant part, a semi-solid of plant part, and/or a powder extract of plant part. Examples of anxiety reducing compounds include melatonin, L-tryptophan, Amanita Muscaria (aga), Apium Graveolens (celery), Aquilegia Vulgaris (columbine), Artemisia Vulgaris (mugwort), Atropa Belladonna (belladonna), Avena Sativa (oats), Ballota Nigra (horehound), Betonica Officinalis (wood betony), Culluna Vulgaris (heather), Cannabis Sativa (marijuana), Capsella Bursa Pastoris (shepherd's purse), Chamaemelum Nobile (Roman chamomile), Cinnamomum Camphora (camphor tree), Citrus Aurantium (bitter orange), Convallaria Majalis (lily-of-the-valley), Corydalis Caba (corydalis), Cyclamen Europaeum (cyclamen), Cypripedium Calceolus (nerve root), Cytisus Scoparius (broom), Drimia Maritima (squill), Eschscholtzia Californica (California poppy), Fragaria Vesca (strawberry leaf), Galium Odorata (woodruff), Humulus Lupulus (hops), Hypericum Perforatum (St. John's wort), Ilex Paraguariensis (mate), Lavandula Angustifolia (lavender), Leonurus Cardiaca (motherwort), Lycopus Virginicus (bugleweed), Matricaria Chamomile L. (German chamomile), Matricaria recutita L. (Hungarian chamomile), Melissa Officinalis (lemon balm), Nepeta Cataria (catnip), Paris Quadrifolia (herb Paris), Passiflora Incarnata (passion flower), Piper Methysticum (kava-kava), Piscidia Piscipula (Jamaica dogwood), Primula Elatior (primrose), Prunus Serotina (black cherry), Rauwolfia Serpentina (rauwolfia), Scutellaria Lateriflora (scullcap), Selenicereus Grandiflorus (night-blooming cereus), Strychnos Nux Vomica (nux vomica), Syzygium Cumini (jambolan), Valeriana Officinalis (valerian), Verbena Officinalis (vervain), Veronica Officinalis (speedwell), and Viscum Album (mistletoe). A combination of anxiety reducing compounds may also be used.

Preferably, the anxiety reducing compound is selected from Apium Graveolens (celery), Avena Sativa (oats) Chamaemelum Nobile (Roman chamomile), Convallaria Majalis (lily-of-the-valley), Cypripedium Calceolus (nerve root), Hypericum Perforatum (St. John's wort) Lavandula Angustifolia (lavender), Mattricaria Chamomile L. (German chamomile), Nepeta Cataria (catnip), Piper Methysticum (kava-kava), and Valeriana Officinalis (valerian). Most preferably, the anxiety reducing compound is valerian or kava-kava.

As used herein, valerian includes any of the fresh, dried, ground, or powdered parts of Valeriana Officinalis Linne, such as the rhizome, roots, and stolons, any extract or volatile oil obtained from Valeriana Officinalis, and any compounds collectively referred to as valerenic acids. Examples of compounds collectively referred to as valerenic acids are valerenic acid, hydroxyvalerenic acid, acetoxyvalerenic acid, valerenal, valeranone, kessyl glycol, and valepotriates, such as didrovaltrate, valtrate, and isovaltrate. As used herein, kava-kava includes any of the fresh or dried, ground or powdered parts of Piper Methysticum G. Forster, such as the rhizomes and roots, any extract obtained from Piper Methysticum, and any compounds referred collectively as kavalactones or kava pyrones, such as methysticin, dihydromethysticin, kawain, dihydrokawain, yangonin, marindinin, and desmethoxyyangonin.

The amount of anxiety reducing compound in the anti-stress composition is preferably from about 50 mg to about 500 mg based on concentrated or solid extract containing not less than 0.5% of the essential oil of the respective anxiety reducing compound. More preferably, the anxiety reducing compound is present in an amount of from about 100 mg to about 430 mg, and most preferably, in an amount of from about 200 mg to about 300 mg. Preferably, the anxiety reducing compound is present in an amount which reduces anxiety but does not induce sleep, unless of course, the anti-stress composition is administered at a time when sleep is desired.

The mental alertness inducing compound is selected from any compounds that promote blood flow. While not wishing to be bound by any particular theory, the inventors believe that increased blood flow has favorable effects on existent neuropathies, neurological and mental functions. Examples of mental alertness inducing compounds include Anthoxanthum Odoratum (sweet vernal grass), Capsella Bursa Pastoris (sheperd's purse), Clematis Recta (clematis), Coriandrum Sativum (coriander), Cornus Florida (dogwood), Cumimum Cyminum (cumin), Curcuma Domestica (turmeric), Daphne Mezereum (merzereon), Digitalis Purpurea (foxglove), Eleutherococcus senticosus (eleuthero root or Siberian ginseng), Echinacea (purple coneflower), Gelsemium Sempervirens (yellow Jessamine), Ginkgo Biloba (ginkgo), Gossypium Herbaceum (cotton), Hibiscus Abelmoschus (muskmallow), Mentha Arvensis Var. Piperascens (Japanese mint), Mentha Longifolia (English horsemint), Panax Ginsent (ginseng), Paris Quadrifolia (herb Paris), Petasites Hybridus (petasites), Salix Species (white willow), Senecio Jacoboea (ragwort), and Viola Odorata (Garden Violet). A combination of mental alertness inducing compounds may also be used.

Preferably, the mental alertness inducing compound is selected from Eleutherococcus Senticosus (eleuthero root or Siberian ginseng), Echinacea (purple coneflower), Ginkgo Biloba (ginkgo), and Panax Ginsent (ginseng). Most preferably, the mental alertness inducing compound is gingko.

The amount of mental alertness inducing compound in the anti-stress composition is preferably from about 20 mg to about 600 mg, based on concentrated or solid extract of the respective mental alertness inducing compound. More preferably, the amount of mental alertness inducing compound is from about 40 mg to about 400 mg, most preferably, from about 60 to about 200 mg.

A food and/or pharmaceutical processing aid is optionally included in the anti-stress compositions to assist in binding all components together, and thereby ensure final product texture, consistency, and, if applicable, dispersability in aqueous media. Examples of processing aids include phosphatides, phospholipids, gelatins, gums such as gum arabic, carrageenan, guar gum, locust bean gum, pectins, and cellulose derivatives. Other food processing aids include those with desirable wetting, lubricating, emulsifying, gelling, swelling, or penetrating properties. Of these, lecithin, either alone or in combination with one or more gums or gelatins may be desirable.

Additional ingredients which may be added to the anti-stress composition include natural and/or artificial ingredients such as vitamins, botanicals, supplements, minerals, trace elements, amino acids (e.g., L. tryptophan), antiflatulents such as simethicone, herbs, fiber, flavorants, enzymes, fillers, buffers, colorants, dyes, sweeteners, pharmaceutical active compounds, antioxidants, medicaments, preservatives, electrolytes, glidants, disintegrates, lubricants, and carrier materials such as polysaccharides. A combination of additional ingredients may also be used. Such ingredients are known to those skilled in the art and preferably are used in the anti-stress compositions in an amount that corresponds to an amount generally recognized as both safe and effective by the United States Food & Drug Administration. For those additional ingredients for which no RDA and/or DV (daily value) has been officially promulgated, then an amount generally accepted in the art as both safe and efficacious may be utilized.

The vitamins are preferably those that are directed to the overall reduction of stress of the user. Accordingly, the vitamin B complex may be selected. This group preferably includes thiamine (B₁) (available as thiamine hydrochloride and thiamine mononitrate), riboflavin (B₂), different chemical forms of what is now considered to be B₃, different chemical forms of what is now considered to be B₅, pyridoxine HCl (B₆) and cyanocobalamin (B₁₂).

Other vitamins may be selected such as vitamin C, E, or B₁₂. It is noted that the antineuritic vitamin thiamine has particular application in reducing emotional hypersensitivity, muscular weakness and fatigue. Riboflavin is important in tissue respiration. Pyridoxine is essential for the dehydration and desulfydration of amino acids and for the normal metabolism of tryptophan. It also appears to be related to fat metabolism. Beneficial properties have also been attributed to the use of folic acid.

Examples of botanicals are Zingiber officinale Roscoe (ginger), tiana lutea (gentianaceae or gentian), Centaurium erythrae Rafn (centaury), Swertia chirata (bitterstick), Menyanthes trifoliata (bogbean), Aftermisia absinthium (Worm wood), Rubis fruiticousus (blackberry leaves or fruit), Rubis family (Blackberry root), Vaccinium corymbosum or V. myrtillus (blueberry leaves), Rubis idaeus or R. strigosus (Raspberry leaves), Mentha x piperita (peppermint), Matricaria recutita and Matricaria chamomilla or Chamomilla recutitia (chamomile), Pimpinella anisum (anise), Carum carvi (caraway), Coriandrum sativum (coriander), Foeniculum vulgare (fennel), Acorus calamus (calamus), Curcuma domestica or C. Ionga, C. zanthorrhiza, C. zedoaria (turmeric), Peumus boldus (boldo), Taraxacum officinale (dandelion), and Glycyrrhiza glabra or G. glabra (licorice)

A variety of minerals may also be added. Zinc oxide, zinc gluconate, zinc sulfate, zinc citrate and/or zinc as aminoate complex may be used in lieu of or in addition to other minerals. Zinc enhances the body's immune system and this enhancement is believed to be beneficial against various pathogens including, it is further believed, Helicobacter pylori (previously called Campylobacter pyloi), the bacteria commonly associated with gastritis and the formation of ulcers. In addition to assisting in the healing of the body, these minerals also provide supplementary amounts of calcium, magnesium and zinc, all necessary metals to maintain body health and metabolism. Furthermore, the minerals and vitamins promote easier and more complete absorption of nutrients by the body, while not changing the pH of the digestive tract which is a common reaction to inorganic salts.

Preferably, the fiber is one or more sources of edible fiber. As that term is used herein, “edible fiber” refers to any source of roughage that is capable of being ingested and processed without harm by animals, in particular humans. Fiber from whatever source is therefore suitable, especially plant matter, such as bran from oats, wheat, corn and rice etc., as well as cellulosic, pectin and gum materials. Husk material may also be preferred. The term “fiber” includes both “soluble” and “insoluble” fiber.

Sweeteners can be chosen from the list of saccharide material available as the carrier component, or can be different materials from those comprising the carrier material, heretofore described. The sweetener is added primarily to provide the anti-stress composition with a palatable sweet taste and flavor. Sweeteners can include mono-, di-, tri- and polysaccharide materials, either alone or in combination, and their related oligomers. Invert sugar, sucrose, fructose, maltose, dextrose, polydextrose, polydextrin, glucose (corn syrup), maltodextrin (corn syrup solids) etc. are just some examples of suitable sweeteners. Additional sweeteners include saccharin, aspartame, acesulfame, sucralose, sorbitol, mannitol, maltitol, and xylitol.

The following nonlimiting examples illustrate further aspects of the invention.

### EXAMPLE 1

An anti-stress composition was prepared by combining valerian, gingko biloba, and calcium carbonate. The valerian was commercially available as valerian root capsule from Sundown Herbals. Each capsule contained 100 mg valerian extract root that is standardized to 0.8% valerenic acid (0.8 mg); and valerian Indian root (430 mg). Two capsules of valerian were administered in a single dosage.

The ginkgo was commercially available as gingko biloba capsule from Sundown Herbals. Each capsule contained ginkgo biloba extract (leaf) 40 mg standardized to 24%; ginkgo flavone glycosides, 9.6 mg., standardized to 6%; terpene lactones 2.4 mg; plus ginkgo biloba (leaf) 410 mg. One capsule of ginkgo was administered.

The calcium carbonate was commercially available as MAALOX® antacid, calcium carbonate chewable tablets from Novartis. Each tablet contained 600 mg. of calcium carbonate. The tablet also contained aspartame, colloidal silicon dioxide, crosarmelose sodium, dextrose, magnesium stearate, maltodextrin, mannitol, and pregelatinized starch. One tablet of calcium carbonate was administered.

### EXAMPLE 2

### Evaluation of Anti-Stress Composition Prepared in Example 1.

The anti-stress composition prepared in Example 1 was administered to three people approximately 30 minutes prior to an exam, meeting, or speaking engagement. Prior to ingesting the anti-stress composition and approximately 15 to 20 minutes after ingesting the anti-stress composition, each person recorded (1) their stress level on a scale of 1 to 5 wherein 5 represented a high level of stress and 1 represented no stress; and (2) their level of alertness on a scale of 1 to 5 wherein 5 represented extreme alertness and 1 represented lethargy. The test results are summarized in Table I.

TABLE I

| Stress Level | Stress Level | Alertness Level | Alertness Level |
|---|---|---|---|
| (Before) | (After) | (Before) | (After) |
| 5 | 1 | 3 | 3 |
| 4 | 1 | 4 | 2 |
| 5 | 2 | 4 | 2 |

The test results in Table I clearly show that the anti-stress compositions of the invention reduce stress in the user within about 15 minutes, however, the level of alertness in each of the three people remained constant or decreased within about 15 minutes after ingesting the anti-stress composition.

### EXAMPLE 3

### Preparation of Anti-Stress Composition.

An anti-stress composition was prepared according to the formula set forth in Example 1, except that 1 capsule of valerian was administered instead of two capsules.

### EXAMPLE 4

### Evaluation of Anti-Stress Composition Prepared in Example 3.

The anti-stress composition prepared in Example 3 was administered to three people approximately 30 minutes prior to an exam or speaking engagement. Prior to ingesting the anti-stress composition and approximately 15 to 20 minutes after ingesting the anti-stress composition, each person recorded (1) their stress level on a scale of 1 to 5 wherein 5 represented a high level of stress and 1 represented no stress; and (2) their level of alertness on a scale of 1 to 5 wherein 5 represented extreme alertness and 1 represented lethargy. The test results are summarized in Table II.

TABLE II

| Stress Level | Stress Level | Alertness Level | Alertness Level |
|---|---|---|---|
| (Before) | (After) | (Before) | (After) |
| 5 | 1 | 3 | 5 |
| 4 | 1 | 3 | 4 |
| 5 | 2 | 4 | 4 |

The test results in Table II clearly show that the anti-stress composition of the invention which contain a critical amount of antacid, anxiety reducing compound, and mental alertness inducing compound simultaneously provide the user with a considerable sense of well-being by providing relief from excess gastrointestinal acidity, reducing anxiety, and increasing mental alertness or thinking ability. The overall sense in the user is that stress is reduced emotionally as well as physically.

While the invention has been described with particular reference to certain embodiments thereof, it will be understood that changes and modifications may be made by those of ordinary skill within the scope and spirit of the following claims:

What is claimed is:

1. An orally administered composition to relieve stress comprising from about 100 mg to about 2000 mg of at least one antacid, about 50 mg to about 500 mg of at least one anxiety reducing compound wherein the amount of anxiety reducing compound is based on a concentrated extract containing not less than 0.5% of the essential oil of the respective anxiety reducing compound, and about 20 mg to about 600 mg of at least one mental alertness inducing compound wherein the amount of the mental alertness inducing compound is based on a concentrated extract of the respective mental alertness inducing compound.

2. An orally administered composition to relieve stress comprising from about 200 mg to about 1200 mg of at least one antacid, about 100 mg to about 430 mg of at least one anxiety reducing compound wherein the amount of anxiety reducing compound is based on a concentrated extract containing not less than 0.5% of the essential oil of the respective anxiety reducing compound, and about 40 mg to about 400 mg of at least one mental alertness inducing compound wherein the amount of the mental alertness inducing compound is based on a concentrated extract of the respective mental alertness inducing compound.

3. An orally administered composition to relieve stress comprising from about 400 mg to about 800 mg of at least one antacid, about 200 mg to about 300 mg of at least one anxiety reducing compound wherein the amount of anxiety reducing compound is based on a concentrated extract containing not less than 0.5% of the essential oil of the respective anxiety reducing compound, and about 60 mg to about 200 mg of at least one mental alertness inducing compound wherein the amount of the mental alertness inducing compound is based on a concentrated extract of the respective mental alertness inducing compound.

4. The composition according to claim 1 wherein the antacid is selected from the group consisting of aluminum carbonate, aluminum hydroxide (or as aluminum hydroxide-hexitol stabilized polymer, aluminum hydroxide-magnesium hydroxide codried gel, aluminum hydroxide-magnesium trisilicate codried gel, aluminum hydroxide-sucrose powder hydrated), aluminum phosphate, aluminum hydroxy carbonate, dihydroxy aluminum sodium carbonate, aluminum magnesium glycinate, dihydroxy aluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, magnesium trisilicate, sodium bicarbonate, potassium bicarbonate, glycine, dried milk solids, sodium carbonate, potassium carbonate, sodium potassium tartrate, and combinations thereof.

5. The composition according to claim 4 wherein the antacid is selected from the group consisting of aluminum hydroxide, calcium carbonate, magnesium carbonate, and magnesium hydroxide.

6. The composition according to claim 1 wherein the anxiety reducing compound is selected from the group consisting of herbal compounds, nonherbal compounds, and combinations thereof.

7. The composition according to claim 6 wherein the anxiety reducing compound is selected from the group consisting of melatonin, L-tryptophan, Amanita Muscaria (aga), Apium Graveolens (celery), Aquilegia Vulgaris (columbine), Artemisia Vulgaris (mugwort), Atropa Belladonna (belladonna), Avena Sativa (oats), Ballota Nigra (horehound), Betonica Officinalis (wood betony), Culluna Vulgaris (heather), Cannabis Sativa (marijuana), Capsella Bursa Pastoris (shepherd's purse), Chamaemelum Nobile (Roman chamomile), Cinnamomum Camphora (camphor tree), Citrus Aurantium (bitter orange), Convallaria Majalis (lily-of-the-valley), Corydalis Caba (corydalis), Cyclamen Europaeum (cyclamen), Cypripedium Calceolus (nerve root), Cytisus Scoparius (broom), Drimia Maritima (squill), Eschscholtzia Californica (California poppy), Fragaria Vesca (strawberry leaf), Galium Odorata (woodruff), Humulus Lupulus (hops), Hypericum Perforatum (St. John's wort), Ilex Paraguariensis (mate), Lavandula Angustifolia (lavender), Leonurus Cardiaca (motherwort), Lycopus Virginicus (bugleweed), Matricaria chamomile L. (German chamomile), Matricaria recutita L. (Hungarian chamomile), Melissa Officinalis (lemon balm), Nepeta Cataria (catnip), Paris Quadrifolia (herb Paris), Passiflora Incamata (passion flower), Piper Methysticum (kava-kava), Piscidia Piscipula (Jamaica dogwood), Primula Elatior (primrose), Prunus Serotina (black cherry), Rauwolfia Serpentina (rauwolfia), Scutellaria Lateriflora (scullcap), Selenicereus Grandiflorus (night-blooming cereus), Strychnos Nux Vomica (nux vomica), Syzygium Cumini (jambolan), Valeriana Officinalis (valerian), Verbena Officinalis (vervain), Veronica Officinalis (speedwell), Viscum Album (mistletoe), and combinations thereof.

8. The composition according to claim 7 wherein the anxiety reducing compound is selected from the group consisting of Apium Graveolens (celery), Avena Sativa (oats) Chamaemelum Nobile (Roman chamomile), Convallaria Majalis (lily-of-the-valley), Cypripedium Calceolus (nerve root), Hypericum Perforatum (St. John's wort) Lavandula Angustifolia (lavender), Mattricaria Chamomile L. (German chamomile), Nepeta Cataria (catnip), Piper Methysticum (kava-kava), and Valeriana Officinalis (valerian).

9. The composition according to claim 8 wherein the anxiety reducing compound is valerian.

10. The composition according to claim 9 wherein the anxiety reducing compound is selected from the group consisting of valerenic acid, hydroxyvalerenic acid, acetoxyvalerenic acid, valerenal, valeranone, kessyl glycol, didrovaltrate, valtrate, and isovaltrate.

11. The composition according to claim 8 wherein the anxiety reducing compound is kava-kava.

12. The composition according to claim 11 wherein the anxiety reducing compound is selected from the group consisting of methysticin, dihydromethysticin, kawain, dihydrokawain, yangonin, marindinin, and desmethoxyyangonin.

13. The composition according to claim 1 wherein the mental alertness inducing compound is selected from any compounds that promote blood flow.

14. The composition according to claim 13 wherein the mental alertness inducing compound is selected from the group consisting of Anthoxanthum Odoratum (sweet vernal grass), Capsella Bursa Pastoris (sheperd's purse), Clematis Recta (clematis), Coriandrum Sativum (coriander), Cornus Florida (dogwood), Cumimum Cyminum (cumin), Curcuma Domestica (turmeric), Daphne Mezereum (merzereon), Digitalis Purpurea (foxglove), Eleutherococcus senticosus (eleuthero root or Siberian ginseng), Echinacea (purple coneflower), Gelsemium Sempervirens (yellow Jessamine), Ginkgo Biloba (ginkgo), Gossypium Herbaceum (cotton), Hibiscus Abelmoschus (muskmallow), Mentha Arvensis Var. Piperascens (Japanese mint), Mentha Longifolia (English horsemint), Panax Ginsent (ginseng), Paris Quadrifolia (herb Paris), Petasites Hybridus (petasites), Salix Species (white willow), Senecio Jacoboea (ragwort), Viola Odorata (garden violet), and combinations thereof.

15. The composition according to claim 14 wherein the mental alertness inducing compound is selected from the group consisting of Eleutherococcus Senticosus (eleuthero root or Siberian ginseng), Echinacea (purple coneflower), Ginkgo Biloba (ginkgo), and Panax Ginsent (ginseng).

16. The composition according to claim 15 wherein mental alertness inducing compound is ginkgo.

17. The composition according to claim 1 further comprising a food processing aid.

18. The composition according to claim 17 wherein the food processing aid is selected from the group consisting of a proteinaceous material, gum, phosphatide, phospholipid, carbohydrate, and combinations thereof.

19. The composition according to claim 1 further comprising a carrier material.

20. The composition according to claim 19 wherein the carrier material is selected from the group consisting of mono-, di-, tri- and polysaccharide materials, and their related oligomers, and combinations thereof.

21. The composition according to claim 20 wherein the carrier material is selected from the group consisting of invert sugar, sucrose, fructose, maltose, dextrose, polydextrose, polydextrin, glucose, and maltodextrin.

22. The composition according to claim 1 further comprising an additional ingredient selected from the group consisting of a vitamin, botanical, supplement, mineral, trace element, amino acid, antiflatulent, herb, fiber, flavorant, enzyme, filler, buffer, colorant, dye, sweetener, pharmaceutical active compound, antioxidant, medicament, preservative, electrolyte, glidant, disintegrate, lubricant, and combinations thereof.

23. The composition according to claim 22 wherein the botanical is selected from the group consisting of Zingiber Officinale Roscoe (ginger), Tiana Lutea (gentianaceae or gentian), Centaurium Erythrae Rafn (centaury), Swertia Chirata (bitterstick), Menyanthes Trifoliata (bogbean), Artermisia Absinthium (worm wood), Rubis Fruiticousus (blackberry leaves or fruit), Rubis family (blackberry root), Vaccinium Corymbosum or V. Myrtillus (blueberry leaves), Rubis Idaeus or R. Strigosus (raspberry leaves), Mentha x Piperita (peppermint), Matricaria Recutita and Matricaria Chamomilla or Chamomilla Recutitia (chamomile), Pimpinella Anisum (anise), Carum Carvi (caraway), Coriandrum Sativum (coriander), Foeniculum Vulgare (fennel), Acorus Calamus (calamus), Curcuma Domestica or C. Longa, C. Zanthorrhiza, C. Zedoaria (turmeric), Peumus Boldus (boldo), Taraxacum Officinale (dandelion), Glycyrrhiza Glabra or G. Glabra (licorice), and combinations thereof.

24. A method to solve behavioral problems in animals including humans comprising orally administering to the animal a composition comprising from about 100 mg to about 2000 mg of at least one antacid, about 50 mg to about 500 mg of at least one anxiety reducing compound wherein the amount of anxiety reducing compound is based on a concentrated extract containing not less than 0.5% of the essential oil of the respective anxiety reducing compound, and about 20 mg to about 600 mg of at least one mental alertness inducing compound wherein the amount of the mental alertness inducing compound is based on a concentrated extract of the respective mental alertness inducing compound.
